(19) United States
(12) Patent Application Publication
(10) Pub. No.: US 2025/0082670 A1
(43) Pub. Date: Mar. 13, 2025

(54) Livestock Feed Additives to Mitigate the Environmental Impact of Ruminants

(71) Applicant: Alejandro R. Castillo, Merced, CA (US)

(72) Inventor: Alejandro R. Castillo, Merced, CA (US)

(21) Appl. No.: 18/726,705

(22) PCT Filed: Dec. 29, 2022
(86) PCT No.: PCT/US22/82505

§ 371 Date: Jul. 3, 2024

Related U.S. Application Data
(60) Provisional application No. 63/266,375, filed on Jan. 4, 2022.

(51) Int. Cl. A61K 33/00; A23K 10/18; A23K 10/30; A23K 20/111; A23K 20/158; A23K 20/163; A23K 20/22; A23K 20/24; A23K 50/10; A61K 31/01; A61K 31/015; A61K 33/06; A61K 33/08; A61K 35/00; A61K 36/064; A61K 36/185; A61K 36/22; A61K 36/24; A61K 36/258; A61K 36/286; A61K 36/288; A61K 36/31; A61K 36/48; A61K 36/49; A61K 36/77; A61K 36/81; A61K 36/88; A61K 36/8962; A61P 1/14

(52) U.S. Cl. CPC A61K 33/00; A23K 10/18; A23K 10/30; A23K 20/111; A23K 20/158; A23K 20/163; A23K 20/22; A23K 20/24; A23K 50/10; A61K 31/01; A61K 31/015; A61K 33/06; A61K 33/08; A61K 36/064; A61K 36/185; A61K 36/22; A61K 36/24; A61K 36/258; A61K 36/286; A61K 36/288; A61K 36/31; A61K 36/48; A61K 36/49; A61K 36/77; A61K 36/81; A61K 36/88; A61K 36/8962; A61P 1/14; A61K 2035/115

(57) ABSTRACT

Disclosed are ruminant feed additive compositions comprising phytochemicals and/or vegetable extracts containing tannins essential oils, optionally saponins and optionally other phytochemicals, buffered with a plurality of acid-neutralizing calcium, magnesium, potassium, and/or sodium salts having pKa's effective at different ruminal and gastrointestinal pH ranges, where the combination of ingredients and their quantities are selected to provide compositions that improves dietary energy and nitrogen utilization efficiency and are effective to reduce greenhouse gas (GHG) emission from the animals. Also disclosed are methods of reducing GHG emissions from ruminants by feeding an effective amount of the composition in an animal feed.

### CROSS REFERENCE TO RELATED APPLICATION

This application claims priority to U.S. Provisional Patent Application No. 63/266,375 filed Jan. 4, 2022, the disclosure of which is incorporated herein by reference in its entirety.

### FIELD OF THE INVENTION

The invention is related to ruminant animal feed additives, and methods of treating ruminants therewith, that reduce greenhouse gas (GHG) emission from the animals during digestion. These feed additives also improve the animals' production efficiency.

### BACKGROUND

At the Paris Climate Change Conference of 2015, countries all over the world agreed to mitigate the industrial and agriculture impacts of global warming by reducing GHG air emissions. Global agriculture contributes with almost 15% of the total anthropogenic GHG emissions. A major part of agriculture emissions is in the form of methane (CH₄, 44%), while the rest is divided between nitrous oxide (N₂O, 29%) and carbon dioxide (CO₂, 27%), where proportions are expressed in terms of CO₂-equivalent. Mitigation interventions should provide both environmental and economic benefits.

Livestock, in particular, ruminants (e.g., cattle, cows, bison, buffalos, sheep, goats, deer, antelope, camels, llamas, and giraffes) produce greenhouse gases.

Thus, feed additives and methods for ruminants are sought which mitigate the GHG emissions, increase productivity, and thereby contribute to global food security and economic development.

### SUMMARY OF THE DISCLOSURE

The present invention meets these needs.

Methods and feed compositions are disclosed herein for improving an animal's production efficiency and simultaneously reducing GHG air emissions (nitrous oxide, carbon dioxide and methane) by combining phytochemicals and/or vegetable extracts containing tannins and essential oils, optionally with saponins and/or other phytochemicals, and buffers comprising a plurality of acid-neutralizing calcium, magnesium, potassium, and/or sodium salts having pKa's effective at different ruminal and gastrointestinal pH ranges. Different types of plant secondary natural products, such as vegetable extracts or natural phytochemicals can be used effectively as feed additives for livestock to mitigate GHG air emissions, increasing productivity, and contributing to food security and economic development.

One aspect of the invention is directed to a feed additive composition for ruminants that reduces greenhouse gas (GHG) emission from the animals, the composition comprising the following combination of ingredients: a) a plurality of acid-neutralizing buffer salts with pKa's effective at different ruminant gastrointestinal pH ranges; b) one or more tannins; c) one or more essential oils; d) optionally, one or more saponins and/or one or more other phytochemicals; where the combination of ingredients and their quantities are selected to provide a composition that improves nutrient utilization efficiency and reduces GHG emission from the animals when fed in an effective amount in an animal feed. The tannins, essential oils, saponins and other phytochemicals can be components of one or more vegetable extracts, or partially purified species, or substantially purified species.

The plurality of acid neutralizing buffer salts of the composition can include at least one salt with a pKa effective to neutralize acids within the pH range of the rumen, and at least one salt with a pKa effective to neutralize acids within the pH range of the ruminant intestine. The salt with a pKa effective to neutralize acids within the pH range of the rumen can include sodium bicarbonate. The salt with a pKa effective to neutralize acids within the pH range of the ruminant intestine can include calcium carbonate, magnesium carbonate, and/or dolomite (natural calcium magnesium carbonate). The composition can also further include magnesium oxide.

Another aspect of the invention is directed to a method for reducing greenhouse gas (GHG) emissions from ruminant animals, the method comprising feeding a ruminant animal an effective amount of any of the above GHG-reducing compositions.

A further aspect of the invention is directed to a greenhouse gas-reducing ruminant feed-additive comprising at least one vegetable feed material and an effective amount of any of the above GHG-reducing compositions.

### DETAILED DESCRIPTION

### Definitions

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention. The term “about” generally includes up to plus or minus 10% of the indicated number. For example, “about 10%” may indicate a range of 9% to 11%, and “about 20” may mean from 18 to 22. Preferably “about” includes up to plus or minus 6% of the indicated value. Alternatively, “about” includes up to plus or minus 5% of the indicated value. Other meanings of “about” may be apparent from the context, such as rounding off, so, for example “about 1” may also mean from 0.5 to 1.4.

As used herein, the term “substantially pure” means greater than 50%, or greater than 60%, or greater than 70%, preferably, greater than 80%, more preferably greater than 90%, most preferably greater than 95% pure. As used herein, the terms “tannin”, “essential oil”, “saponin” and “other phytochemicals” are defined in detail below.

### Tannins:

Tannins are a class of astringent, polyphenolic compounds that bind to and precipitate proteins, amino acids and other organic compounds such as alkaloids. Tannins constitute the fourth most abundant biochemicals produced by vascular plant tissues, after cellulose, hemicellulose and lignin, representing a significant portion of the terrestrial biomass. Because they are complex and energetically costly molecules to synthesize, their widespread occurrence and abundance has suggested that tannins play an important role in plant function and evolution. Tannins are well-known either as astringent and antioxidant components or as ill-defined plant substance that were historically used to convert animal skins into leather.

The term polyphenolic compounds refers to a wide class of plant secondary metabolites having a phenolic moiety, bearing at least one hydroxyl substituent. Tannin compounds can range from simple phenolics (e.g., ellagic and gallic acids), to dimeric or oligomeric compounds (e.g., procyanidins, lignans), or to polymeric compounds with high molecular weight. Tannins are the end products of energy demanding and extensive biosynthetic pathways, indicating that they play an important role in plant metabolism. They can be broadly divided into two groups, viz., hydrolyzable and condensed tannins, depending on their structure. Hydrolysable tannins contain a central polyol esterified with gallic acid molecules. They can be further divided into three groups: simple gallic acid derivatives, gallotannins, and ellagitannins. Condensed tannins are the second most abundant polyphenols after lignins with two or more flavan-3-ol monomeric units. The most common flavan-3-ol subunits are characterized based on the number of hydroxyl groups, that give it the high reactive capacity of condensed tannins.

The main commercial tannins useful for animal nutrition come from three tree species, Quebracho (Schinopsis sp.) and Chestnut (Castanea sp.), classified as condensed and hydrolyzable tannins, respectively; and a legume tree, Tara (Caesalpinia spinosa) which provides a condensed tannin. Tannins companies are cutting only old trees, and the rate of extraction and replanting is controlled to maintain the sustainability of these forest systems. Tannins come from trunk wood of the trees. Modern industrial extraction processes are used without solvents or toxic chemicals. The production of wood tannins from old trees is very efficient, maintaining tannin quality without major chemical changes when different parts of the plant are harvested, and is also not affected by the season of the year or environmental factors.

Tannin polyphenols can range from simple phenolics (e.g., ellagic and gallic acids), to dimeric or oligomeric compounds (e.g., procyanidins, lignans), to polymeric compounds with high molecular weight as condensed and hydrolysable tannins (e.g. extracts of quebracho, chestnut, tara, birdsfoot, lotus, sainfoin, onobrychis, mimosa, hedysarum, proanthocyanidin, acacia, etc.).

With regard to utility in a composition for animal nutrition, the phytochemicals in general, and the tannins in particular, function most effectively in a pH range of 6.0 to 6.5 when different inorganic ions are added to the solution (Ca, Mg, K, and Na), which is the main objective of the buffer (pH control and adding cations). Thus, a pH of 6.0 to 6.5 is the ideal range of rumen pH to maximize the tannins' mode of action. Further, components such as tannins, essential oils, saponins and other phytochemicals are not foreign to the ruminant diet; ruminants have been eating phytochemicals for millions of years in the form of various plants, e.g., grass, legumes, trees, and bushes.

### Essential Oils:

An essential oil is the concentrated hydrophobic liquid extract from a plant containing the volatile chemical compounds from that plant. Essential oils are also known as volatile oils, ethereal oils, aetheroleum, or simply as the oil of the plant from which they were extracted, e.g., “oil of clove”. An essential oil is “essential” in the sense that it contains the “essence of” the plant's fragrance, i.e., the characteristic fragrance of the plant from which it was derived.

Essential oils are generally composed of terpenes and secondary metabolites of phenylpropene, and include essential oils of citrus, e.g., orange or lemon [limonene]; garlic, capsicum, lycopene, cumin, thymol, coriander, eugenol, cinnamon, horseradish, curcuma, peppermint, spearmint, Mentha arvensis, cedarwood, sandalwood, Eucalyptus globulus, Litsea cubeba, and clove.

### Saponins:

Saponins are found in various botanical families, and are classified as triterpenoids, steroids, and alkaloids extracted from natural sources such as yucca, quillaja, soapberry or soapnut, oleander, ginseng or red ginseng, and sesbania. The saponins can be incorporated in the GHG-reducing compositions in the form of extracts from any of the above, for example yucca extracts.

### Other Phytochemicals:

Phytochemicals are chemicals of plant origin, produced by plants through primary or secondary metabolism. Major categories of phytochemicals, aside from polyphenols, include carotenoids, which include, for example, lycopene. “Other phytochemicals” can include extracts of dandelion (Taraxacum officinale), mustard (Brassica juncea) and safflower (Carthamus tinctorius), and some natural sources can include various nut extracts from Anacardium sp., common name “cashew nuts”, from Brazil, India, Colombia, and various African countries.

Representative ranges of the above components in the GHG-reducing compositions are presented in the following table:

Dietary Dry Matter
basis ranges (%)

| Component | min | max |
|---|---|---|
| Tannin blends | 0.04 | 0.50 |
| Essential oil blends | 0.004 | 0.02 |
| Saponin blends | 0 | 0.02 |
| Other phytochemical blends | 0 | 0.50 |

### Ruminant Buffers:

Ruminant physiology is unique. These grazing, browsing or non-grazing system mammals are able to acquire nutrients from plant-based food by fermenting it in a specialized multi-compartmented stomach prior to digestion, with the fermentation occurring principally through microbial actions. This is a continuous fermentation process where the maintenance and control of rumen pH is critical.

The average ideal rumen pH is between 6.0 and 6.5 to maximize fiber digestion and maintain the continuous fermentation system. A pH lower than 5.8 increases the risk of sub-clinical acidosis and below 5.5 is considered an acidosis condition. Animals may die when rumen pH falls below 5.0. In contrast, digestive enzymes in the small intestine are ineffective below pH 6.8.

Combinations of buffer salts, designed to be differentially active in the rumen and small intestine at their respective pH optima, are employed to maximize the efficacy of the feed-additive compositions of the invention. Thus, at least one acid neutralizing salt has a pKa effective to neutralize acids within the pH range of the rumen, and at least one acid neutralizing salt has a pKa effective to neutralize acids within the pH range of the ruminant intestine. Examples of acid neutralizing salts include sodium, calcium and magnesium carbonates, calcium and magnesium oxides, and sodium, calcium and magnesium phosphates. Sodium carbonates include sodium bicarbonate (NaHCO₃), and sodium sesquicarbonate dihydrate.

Sodium bicarbonate is effective to neutralize acidic rumen pH. In contrast, calcium carbonate, magnesium carbonate and dolomite are effective to neutralize acidic pH in the ruminant intestine. Magnesium oxide and inorganic acid absorbing materials (aluminosilicates) function to neutralize acidic pH in both the rumen and the ruminant intestine. The active probiotic culture helps to protect lactic acid-consuming rumen bacteria in a symbiotic process.

Sodium bicarbonate is widely used by feedlots and intensive dairy production systems as a ruminant feed amendment to prevent sub-clinical acidosis. The pKa of sodium bicarbonate is only effective to partially neutralize acids in the rumen and it will not neutralize acid in the intestine. Further, from the environmental point of view, sodium bicarbonate has high sodium content. This results in soil salinization and underground water contamination in intensive animal production systems. Thus, the mixture of buffer salts having different cations, found in the present feed compositions is 1) lower in sodium content than pure sodium bicarbonate, and 2) unlike sodium bicarbonate, the salt mixture is capable of neutralizing acids over the variety of pH ranges found in ruminant gastrointestinal systems.

Optionally, the composition can also contain an active probiotic culture. Such compositions contain between about 1 and about 5 wt %, preferably between about 2 and about 4 wt % and more preferably about 3 wt % of the active probiotic culture. The active probiotic culture preferably stimulates the growth and concentration of lactic acid consuming rumen microorganisms, and is preferably an active yeast culture. The active yeast culture is preferably a Saccharomyces, and even more preferably Saccharomyces cerevisiae. One example of a suitable S. cerevisiae yeast strain is the CNCM (Pasteur Institute) 1-1077 strain of S. cerevisiae.

Optionally, the composition can also include an acid-absorbing inorganic material, such as bentonite, zeolite, and some other clays and volcanic ashes. Such compositions contain up to about 30 wt % of the acid-absorbing inorganic material. An inorganic material level between about 10 and about 30 wt % is preferred, with a level of about 15 to about 25 wt % being more preferred. Bentonite is a particularly useful acid-absorbing inorganic material because it also decreases the rate of passage of rumen digesta and increases fiber digestion.

One aspect of the invention is directed to a feed additive composition for ruminants that reduces greenhouse gas (GHG) emission from the animals, the composition comprising the following combination of ingredients: a) a plurality of acid-neutralizing buffer salts with pKa's effective at different ruminant gastrointestinal pH ranges; b) one or more tannins; c) one or more essential oils; d) optionally, one or more saponins and/or one or more other phytochemicals; where the combination of ingredients and their quantities are selected to provide a composition that reduces GHG emission from the animals when fed in an effective amount in an animal feed. The tannins and/or essential oils of the composition can be contained in a vegetable extract. The saponins and/or other phytochemicals of the composition can be contained in a vegetable extract, which may be the same or different from the tannin/essential oil-containing extract.

The tannins of the composition can be present in isolated, substantially pure form, or alternatively can be present in one or more of the following vegetable extracts: Quebracho, Chestnut, Tara, Acacia. The essential oil can be present in isolated, substantially pure form, or alternatively can be present in one or more of the following vegetable extracts: Limonene, Garlic, Capsicum, Lycopene. The saponins can be present in isolated, substantially pure form, or alternatively can be present in one or more of the following vegetable extracts: Yucca, Quillaja, Soapberry or Soapnut, Oleander, Ginseng. The “other phytochemicals” can be present in isolated, substantially pure form, or alternatively can be present in one or more of the following vegetable extracts: Cashew nuts, Dandelion, Mustard and Safflower. When present in vegetable extracts, the desired tannin, essential oil, saponin and other phytochemical components are typically present in about 40 to about 90 wt %.

The plurality of acid neutralizing buffer salts of the composition can include at least one salt with a pKa effective to neutralize acids within the pH range of the rumen, and at least one salt with a pKa effective to neutralize acids within the pHrange of the ruminant intestine. The salt with a pKa effective to neutralize acids within the pH range of the rumen can include sodium bicarbonate. The salt(s) with a pKa effective to neutralize acids within the pH range of the ruminant intestine can include calcium carbonate, magnesium carbonate, and/or dolomite. The salt with a pKa effective to neutralize acids within the pH range of the rumen can be present in about 10 wt % to about 30 wt % based on the composition. The salt(s) with a pKa effective to neutralize acids within the pH range of the ruminant intestine can be present in about 30 wt % to about 70 wt % based on the composition. Thus, sodium bicarbonate can be present in the GHG-reducing compositions in about 10 to about 30 wt %; calcium carbonate can be present in about 10 to about 20 wt %; and dolomite can be present in about 20 to about 50 wt %.

The composition can further include magnesium oxide. The magnesium oxide can be present in about 5 wt % to about 15 wt % based on the composition. The composition can also further include bentonite in about 10 to about 30 wt %. The composition can further include an active probiotic culture in about 1 to about 5 wt %. The composition preferably includes an active Saccharomyces cerevisiae yeast culture in about 1 to about 5 wt %.

The acid neutralizing buffer salts of the composition preferably constitute between about 66 wt % and about 90 wt % based on the composition.

Another aspect of the invention is directed to a method for reducing greenhouse gas (GHG) emissions from ruminant animals, the method comprising feeding a ruminant animal an effective amount of any of the above GHG-reducing compositions. The composition can be fed to the ruminant animal daily. When the ruminant animal weighs over 100 kg, it is fed between about 25 g and about 50 g of the composition per 100 kg of body weight. When the ruminant animal weighs less than 100 kg, it is fed between about 2.5 g and about 10 g of the composition per 10 kg of body weight.

A further aspect of the invention is directed to a greenhouse gas-reducing ruminant feed comprising at least one vegetable feed material and an effective amount of any of the above GHG-reducing compositions. The vegetable feed material of the GHG-reducing ruminant feed composition can be a grain or feed concentrate under grazing conditions.

The present invention also includes a ruminant feed containing at least one vegetable feed material and between about 1 and about 2.5 wt % on a dry solid basis of the GHG-reducing composition of the present invention. About 1.2% by weight of the total dry matter intake or about 2% by weight of the total concentrate intake is preferred.

The GHG-reducing compositions of the present invention may be conveniently fed to a ruminant admixed with a conventional ruminant feed. The feeds are typically vegetable materials edible by ruminants, such as legume hay, grass hay, corn silage, grass silage, legume silage, corn grain, oats, barley, distiller's grain, brewer's grain, soya bean meal, cottonseed meal, etc. Pre-mixes, (e.g., minerals and vitamins, concentrates or grains, etc.) are preferred. For animals in free stalls or dry lot pens, the GHG-reducing composition should be also pre-mixed with other ingredients in a Total Mixed Ration. The composition can be mixed with grains for supplementing grazing animals. Because of its characteristics (dry fine powder) it is not recommended to offer this product alone.

Desirably, the amount of the GHG-reducing composition in a feed ration admixture does not exceed about 1.2 wt % of the dry matter content of the total daily ration and is preferably between about 0.8 and about 1.8% of the dry matter content of the daily ration. There is no particular lower limit for the amount of the composition to be added to the ruminant feed, although in practice amounts below about 0.6 wt % of the dry matter content is too small to provide significant beneficial effects.

For ruminants weighing over 100 kg (young or adult cows, etc.), between about 25 and about 50 g per 100 kg of body weight of the GHG-reducing composition should be administered, and preferably about 40 g per 100 kg of body weight or 2% of the total concentrate intake including grain content in the silage (e.g. corn). For ruminants weighing less than 100 kg (goats, sheep, deer, etc.), from about 2.5 to about 10 g per 10 kg of body weight should be administered, and preferably about 5 g per 10 kg.

The GHG-reducing compositions are intended to be fed to ruminants on a daily basis, preferably no less than 7 days a week.

### EXAMPLES

The following examples are intended to be illustrative of the present invention, and in no way limit its scope.

### Example 1. Typical Composition for GHG-Reducing Ruminant Feed Additive

The following composition ranges are based on diets for small animals (sheep or goats) and high producing dairy cows (>700 kg body weight). All components are blended together, the product is dry, and can be mixed easily into a feed premix or ration.

| | International | Ranges |
|---|---|---|
| Feed-additive Composition | Feed Number | (wt %) |
| Dolomite | 6-02-633 | 20-50 |
| Alumino Silicates (Bentonite) | na* | 10-30 |
| Sodium Bicarbonate | 6-04-272 | 10-30 |
| Calcium Carbonate | 6-02-632 | 10-20 |
| Magnesium Oxide | 6-02-756 | 5-15 |
| Yeast extracts | Na* | 1-5 |
| Vegetable extracts, blends** | na | 3-20 |

*na = not available
**includes tannins and essential oils, and optionally saponins and/or other phytochemicals

Numerous combinations of the features set forth above can be utilized without departing from the present invention as set forth in the claims. It will be understood by those of ordinary skill in the art that various modifications can be made without departing from the spirit and scope of the present invention. Therefore, it should be clearly understood that the various embodiments of the present invention described herein are illustrative only, and are not intended to limit in any way the scope of the present invention.

What is claimed is:

1. A feed additive composition for ruminants that reduces greenhouse gas (GHG) emission from the animals, the composition comprising the following combination of ingredients:
a) a plurality of acid-neutralizing buffer salts with pKa's effective at different ruminant gastrointestinal pH ranges;
b) one or more tannins;
c) one or more essential oils;
d) optionally, one or more saponins and/or one or more other phytochemicals;
wherein the combination of ingredients and their quantities are selected to provide a composition that control rumen and small intestine pH and reduces GHG emission from the animals when fed in an effective amount in an animal feed.

2. The composition of claim 1, wherein the tannins and/or essential oils are contained in a vegetable extract.

3. The composition of claim 1 or 2, wherein the saponins and/or other phytochemicals are contained in a vegetable extract.

4. The composition of any one of claims 1 to 3, wherein the tannins comprise one or more extracts of Quebracho, Chestnut, Tara, and Acacia.

5. The composition of any one of the preceding claims, wherein the essential oils comprise one or more of Limonene, Garlic, Capsicum, and Lycopene.

6. The composition of any one of the preceding claims, wherein the saponins comprise one or more extracts of Yucca, Quillaja, Soapberry, Soapnut, Oleander, and Ginseng.

7. The composition of any one of the preceding claims, wherein the other phytochemicals comprise one or more extracts of Cashew nuts, Dandelion, Mustard and Safflower.

8. The composition of any one of the preceding claims, wherein the plurality of acid neutralizing buffer salts comprises at least one salt with a pKa effective to neutralize acids within the pH range of the rumen, and at least one salt with a pKa effective to neutralize acids within the pH range of the ruminant intestine.

9. The composition of claim 8, wherein the salt with a pKa effective to neutralize acids within the pH range of the rumen comprises sodium bicarbonate.

10. The composition of claim 8 or 9, wherein the salt with a pKa effective to neutralize acids within the pH range of the ruminant intestine comprises calcium carbonate, magnesium carbonate, and/or dolomite.

11. The composition of any one of claims 8 to 10, wherein the salt with a pKa effective to neutralize acids within the pH range of the rumen is present in about 10 to about 30 wt % based on the composition.

12. The composition of any one of claims 8 to 11, wherein the salt with a pKa effective to neutralize acids within the pH range of the ruminant intestine is present in about 30 to about 70 wt % based on the composition.

13. The composition of any one of the preceding claims, further comprising magnesium oxide.

14. The composition of claim 13, wherein the magnesium oxide is present in about 5 to about 15 wt % based on the composition.

15. The composition of any one of the preceding claims, further comprising bentonite.

16. The composition of any one of the preceding claims, further comprising an active probiotic culture.

17. The composition of any one of the preceding claims, further comprising an active Saccharomyces cerevisiae yeast culture.

18. The composition of any one of the preceding claims, wherein the acid neutralizing buffer salts comprise between about 66% by weight and about 90% by weight of the composition.

19. A method for reducing greenhouse gas (GHG) emissions from ruminant animals, the method comprising feeding a ruminant animal an effective amount of the GHG-reducing composition of any one of claims 1 to 18.

20. The method of claim 19, wherein the composition is fed to the ruminant animal daily.

21. The method of claim 19 or 20, wherein the ruminant animal weighs over 100 kg and is fed between about 25 and about 50 g of the composition per 100 kg of body weight.

22. The method of claim 19 or 20, wherein the ruminant animal weighs less than 100 kg and is fed between about 2.5 and about 10 g of the composition per 10 kg of body weight.

23. A greenhouse gas-reducing ruminant feed comprising at least one vegetable feed material and an effective amount of the GHG-reducing composition of any one of claims 1 to 18.

24. The GHG-reducing ruminant feed composition of claim 23, wherein the vegetable feed material is a grain or feed concentrate.
